(19) United States
(12) Patent Application Publication
(10) Pub. No.: US 2011/0293559 A1
(43) Pub. Date: Dec. 1, 2011

(54) CATABOLIC AGENTS

(76) Inventors: Sophie Jane Gilbert, Monmouthshire (GB); Simarjit Kaur Singhrao, Cardiff (GB); Ilyas Mahmoud Khan, Cardiff (GB)

(21) Appl. No.: 13/054,689

(22) PCT Filed: Jul. 10, 2009
(86) PCT No.: PCT/GB2009/001722

§ 371 Date: Aug. 23, 2011

(30) Foreign Application Priority Data
Jul. 18, 2008 (GB) 0813199.7

(51) Int. Cl. A61K 38/20; A61K 38/48; C12N 9/50; C07K 14/525; C07K 14/545; C07K 14/54; A61K 38/19; A61P 19/04

(52) U.S. Cl. 424/85.2; 424/85.1; 424/94.67; 514/8.8; 530/351; 435/219

(57) ABSTRACT

This invention relates to the use of agents which are capable of the catabolism of components of cartilage extracellular matrix to promote cartilage regeneration within cartilage pathologies and to promote cartilage integration within focal defects.

### RELATED APPLICATIONS

This application claims priority to UK patent application No. 0813199.7 filed on 18 Jul. 2008, the disclosures of which are incorporated herein by reference in their entirety.

### FIELD OF THE INVENTION

This invention relates to the use of agents which are capable of the catabolism of components of cartilage extracellular matrix to promote cartilage regeneration within cartilage pathologies and to promote cartilage integration within focal defects.

### BACKGROUND TO THE INVENTION

Cartilage integration is a chronic problem that affects healing of cartilage to cartilage either during the intrinsic healing of focal defects or following cartilage surgical implantation procedures (Hunziker, 2002). The principal insufficiency of cartilage to promote effective healing is that it is avascular therefore injured cartilage that has lesions larger than three millimetres rarely heal to give a hyaline replacement tissue. In place of hyaline cartilage, larger defects that have penetrated through to the subchondral bony plate are filled by hematomas, but even in this situation void filling only occurs sufficiently in defects smaller than six millimetres in diameter.

Migration of mesenchymal stem cells to the hematoma generates, in time, a fibrocartilagenous replacement tissue that is biomechanically inferior to normal hyaline cartilage. The generation of a biomechanical discontinuity causes gradual degeneration of chondrocytes and extracellular matrix the junction of the fibrocartilagenous repair and normal hyaline cartilage (Shapiro et al., 1993). Cell death of chondrocytes causes insufficient maintenance of the extracellular matrix leading to microfractures and larger fissures appearing in the ecm. A proliferative response by surviving chondrocytes results in single large chondrons occupied by multiple clonally-derived chondrocytes. The same degenerative response can also occur when chondrocyte implantation is used to heal focal lesions. Matrix-assisted chondrocyte implantation has evolved to biomechanically stabilise the nascent repair tissue until the extracellular matrix matures to give a more hyaline appearance.

There are many factors that have been described that affect cartilage-cartilage integration. Cellular density at the junction between repair and normal tissue is a primary factor that directly affects integration between cartilages. Cell death inhibits and increased proliferation stimulates cartilage-cartilage integration. It is well known that extracellular matrix generally inhibits cartilage integration; proteoglycans are inhibitory not only to integration but also for chondrocyte migration (Hunziker, 2002).

The most successful methods to induce cartilage integration have involved remodelling the extracellular matrix of articular cartilage, specifically removal of proteoglycans and digestion of the collagen structural framework (Bos et al., 2002; Janssen et al., 2006; van de Breevaart Bravenboer et al., 2004). Trypsin, collagenase and metalloproteinases have been used to degrade the extracellular matrix of cartilage prior to attempting to fuse cartilage surfaces (Bos et al., 2002; Caplan et al., 1997; Obradovic et al., 2001).

Cytokines are soluble or cell surface molecules that mediate cell-cell interactions. With respect to regulation of chondrocyte function, it is possible to classify the cytokines that regulate cartilage remodeling as (i) catabolic cytokines, which act on target cells to increase products that enhance matrix degradation; (ii) anti-catabolic or inhibitory cytokines, which inhibit or antagonize the activity of the catabolic cytokines; and (iii) anabolic cytokines, which act as growth and differentiation factors on chondrocytes to increase synthetic activity.

Catabolic cytokines; interleukin-1 (IL-1), tumour necrosis factor, alpha (TNFα), IL-17, IL-18 and oncostatin-M (OSM) are known to stimulate the degradation of the extracellular matrix through the production of proteolytic enzymes such as metalloprotinases (MMP) and aggrecanases. Tetlow et al showed that catabolic cytokines co-localised with proteolytic enzymes in articular cartilage using immunohistochemical methods (Tetlow et al., 2001).

Whilst specific enzymatic digestion of either the proteoglycan or collagenous components of the extracellular matrix has previously been shown to be useful in accelerating cartilage integration the use of catabolic cytokines causes the resident chondrocytes to remodel the extracellular matrix. As the mode of degradation of the extracellular matrix by the catabolic cytokines is more controlled and organised this method provides an improved alternative to pure enzymatic digestion.

It would appear to go against convention to use a catabolic agent of the cartilage extracellular matrix in a treatment regime for a cartilage pathology. The skilled man would consider such a use would be more detrimental to the cartilage rather than promoting the reparative processes. Although, Englert et al, 2006 showed enhanced cartilage integration when cartilage was treated with 10 pg/ml IL-1β, this result was not consistent at 1 pg/ml or 100 pg/ml. Furthermore, this was only demonstrated in vitro following the incubation of the cartilage for 14 days.

### SUMMARY OF THE INVENTION

We have found that an agent capable of catabolising a component of cartilage, either when used alone or in combination with at least one agent capable of anabolising a component of cartilage, provides an improved therapeutic response in a subject with a cartilage pathology.

The catabolic agent removes the acellular matrix thereby enabling the chondrocytes to migrate unhindered to the site of damage and then initiate the reparative process by re-synthesising components of cartilage.

According to a first aspect of the invention there is provided use of an agent capable of causing the catabolism of a component of cartilage in the preparation of a medicament for use in the treatment of a cartilage pathology in a subject.

According to a second aspect of the invention there is provided a medicament comprising at least one agent capable of the catabolism of at least one component of cartilage extracellular matrix and at least one agent capable of the anabolism of at least one component of cartilage extracellular matrix.

According to a third aspect of the invention there is provided a method of treatment of a cartilage pathology in a subject, the method comprising the steps of;
i) providing a medicament according to the first or second aspect of the invention and;
ii) administering the medicament to the subject.

The administration of the medicament according to the invention results the promotion of cartilage-cartilage integration at the pathological site and/or the promotion of cartilage integration into an implant provided at the site.

Cartilage is classified in three types, elastic cartilage, hyaline cartilage and fibrocartilage, which differ in their relative amounts of collagen, proteoglycan and elastin fibres.

Hyaline cartilage is primarily made up of type II collagen and chondroitin sulphate.

In embodiments of the invention the agent capable of catabolising a component of the cartilage matrix is capable of catabolising or degrading the collagen component. In particular, the type II collagen component.

In embodiments of the invention the agent capable of catabolising a component of cartilage matrix is capable of catabolising or degrading the proteoglycan component. In particular, the chondroitin sulphate component.

The medicament may comprise an agent capable of degrading the collagen component and an agent capable of degrading the proteoglycan component.

In embodiments of the invention the catabolic agent is a cytokine. Specifically a catabolic cytokine, which may also be referred to as a matrix degrading cytokine.

A catabolic cytokine is defined as a cytokine which regulates cartilage function through acting on target cells to increase their expression of proteins that decrease extracellular matrix.

Examples of suitable catabolic cytokines include, but are not limited to, members of the interleukin-1 superfamily, particularly interleukin-1 alpha (IL-1α) and interleukin-1 beta (IL-1β) and isoforms thereof.

In embodiments of the invention the catabolic cytokine is not IL-1β or isoforms thereof at a therapeutically effective dose of about 10 pg/ml.

In embodiments of the invention the catabolic cytokine is not IL-1β or isoforms thereof.

In embodiments of the invention the catabolic cytokine is not IL-1α or isoforms thereof.

It is envisaged that IL-1β is at a therapeutically effective dose of equal to or greater than about 100 pg/ml.

It is envisaged that IL-1β is at a therapeutically effective dose of equal to or greater than about 1 ng/ml.

It is envisaged that IL-1β is at a therapeutically effective dose of equal to or greater than about 10 ng/ml.

It is envisaged that IL-1β is at a therapeutically effective dose in the range of between about 1 ng/ml and 100 ng/ml.

It is envisaged that IL-1β is at a therapeutically effective dose in the range of between about 10 ng/ml and 50 ng/ml.

It is envisaged that IL-1β is at a therapeutically effective dose of about 10, 12.5, 15, 20, 25, 30, 35, 40, 45, 50, 55, 60, 65, 70, 75, 80, 85, 90, 95 or 100 ng/ml.

Other suitable catabolic cytokines include interleukin-18, tumour necrosis factor alpha (TNFα), oncostatin M and isoforms thereof.

It is envisaged TNFα is at a therapeutically effective dose of equal to or greater than about 100 pg/ml.

It is envisaged that TNFα is at a therapeutically effective dose of equal to or greater than about 1 ng/ml.

It is envisaged that TNFα is at a therapeutically effective dose of equal to or greater than about 10 ng/ml.

It is envisaged that TNFα is at a therapeutically effective dose of equal to or greater than about 100 ng/ml.

It is envisaged that TNFα is at a therapeutically effective dose in the range of between about 1 ng/ml and 1 mg/ml.

It is envisaged that TNFα is at a therapeutically effective dose in the range of between about 1 ng/ml and 100 ng/ml.

It is envisaged that TNFα is at a therapeutically effective dose in the range of between about 10 ng/ml and 50 ng/ml.

It is envisaged that TNFα is at a therapeutically effective dose of about 10, 12.5, 15, 20, 25, 30, 35, 40, 45, 50, 55, 60, 65, 70, 75, 80, 85, 90, 95 or 100 ng/ml.

In embodiments of the invention the medicament comprises at least two catabolic cytokines. For example, a combination of interleukin-1 beta (IL-1β) and tumour necrosis factor alpha (TNFα).

It is envisaged that a medicament comprises between about 10 and 100 ng/ml TNFα and between about 10 and 100 ng/ml IL-1β.

It is envisaged that a medicament comprises between about 50 and 100 ng/ml TNFα and between about 10 and 50 ng/ml IL-1β.

It is envisaged that a medicament comprises about 100 ng/ml TNFα and between about 10 and 100 ng/ml IL-1β.

It is envisaged that a medicament comprises about 100 ng/ml TNFα and between about 10 and 50 ng/ml IL-1β.

It is envisaged that a medicament comprises about 100 ng/ml TNFα and between about 10 and 25 ng/ml IL-1β.

It is envisaged that a medicament comprises about 100 ng/ml TNFα and also between about 10 and 15 ng/ml IL-1β.

It is envisaged that a medicament comprises about 100 ng/ml TNFα and between about 10 and 12.5 ng/ml IL-1β.

It is envisaged that a medicament comprises about 100 ng/ml TNFα and about 10, 12.5, 15, 20, 25, 30, 35, 40, 45, 50, 55, 60, 65, 70, 75, 80, 85, 90, 95 or 100 ng/ml IL-1β.

In embodiments of the invention the catabolic agent is an agent which induces the production of a catabolic cytokine. For example interleukin-17 (IL-17) induces the production of many other cytokines such as IL-1β and TNF-α.

In embodiments of the invention the catabolic agent is a proteolytic enzyme capable of the proteolysis of components of the cartilage matrix. In embodiments of the invention the proteolytic enzyme is capable of the proteolysis of the collagen and/or proteoglycan component of cartilage.

In embodiments of the invention the proteolytic enzyme is a matrix metalloproteinase (MMP). For example MMP-13 (collagenase-3) or variants thereof, which is capable of the proteolysis of type II collagen.

In embodiments of the invention the proteolytic enzyme is a disintegrin and metalloproteinase (ADAM). For example ADAMTS4 (aggrecanase-1) or ADAMTS11 (aggrecanase-2).

In further embodiments of the invention the catabolic agent is an activator of the pro-form of a proteolytic enzyme. For example an agent capable of activating pro-MMPs, such as pro-MMP13. Suitable agents include serine proteases such as plasmin, plasma kallikrein and neutrophil elastase. A further suitable agent is retinoic acid.

Retinoic acid induces MMP-13 and ADAMTS4 and can therefore be used to upregulate these proteolytic enzymes.

It is envisaged retinoic acid is at a therapeutically effective dose of equal to or greater than about 100 μM.

It is envisaged that retinoic acid is at a therapeutically effective dose of equal to or greater than about 1 μM.

It is envisaged that retinoic acid is at a therapeutically effective dose of equal to or greater than about 10 μM.

It is envisaged that retinoic acid is at a therapeutically effective dose of equal to or greater than about 100 μM.

It is envisaged that retinoic acid is at a therapeutically effective dose in the range of between about 1 μM and 10 μM

It is envisaged that retinoic acid is at a therapeutically effective dose in the range of between about 1 μM and 100 μM.

It is envisaged that retinoic acid is at a therapeutically effective dose in the range of between about 10 μM and 50 μM.

It is envisaged that retinoic acid is at a therapeutically effective dose of about 10, 12.5, 15, 20, 25, 30, 35, 40, 45, 50, 55, 60, 65, 70, 75, 80, 85, 90, 95 or 100 μM.

It is envisaged that more than one agent capable of the catabolism of at least one component of the cartilage matrix can be combined in the medicament. For example MMP-13 and ADAMTS-4 or ADAMTS-11.

Once the existing acellular matrix has been degraded by the direct or indirect effect of a catabolic agent within the medicament, thereby enabling the migration of chondrocytes into the defect, it is envisaged that the synthesis of a new extracellular matrix can be enhanced by the use of an agent which causes the anabolism/synthesis of at least one component of the cartilage extracellular matrix.

Examples of suitable agents include cytokines which are non-catabolic, as herein defined as encompassing cytokines which are not catabolic and demonstrate either anabolic, modulatory and/or anti-catabolic activity.

Anabolic cytokines are herein defined as proteins which acts as growth or differentiation factors and generally enhance the expression of components of the extracellular matrix. Examples include the bone morphogenetic proteins (BMP) and the Transforming Growth Factor B (TGFβ) family of proteins.

Inhibitory cytokines are herein defined as proteins which directly or indirectly inhibit the function of catabolic cytokines, for example decoy soluble receptors to IL-1.

Modulatory cytokines are herein defined as proteins which antagonise or act as agonists of other cytokines in a context dependent manner, an examples of such a cytokine include IL-6 and IL-11. These act in positive or negative feedback loops to regulate the function of the primary catabolic cytokines.

An example of a suitable anabolic cytokine is TGF-β and isoforms thereof.

An example of a suitable modulatory cytokine is IL-6 and isoforms thereof.

Examples of a suitable anti-catabolic cytokines include IL-10 and activin and isoforms thereof.

It is therefore envisaged in embodiments of the invention that an agent which is capable of promoting the anabolism of at least one component of the cartilage extracellular matrix is either administered in combination with or separately to the agent which promotes the catabolism of at least one component of the cartilage extracellular matrix.

Advantageously the medicament comprises agents capable of promoting both catabolism and anabolism of the collagen component and/or the proteoglycan component of the cartilage extracellular matrix.

For example an anabolic agent can be incorporated into the medicament according to the invention. The anabolic agent can be incorporated into the medicament such that it is released simultaneously with the catabolic agent. Alternatively the anabolic agent can be incorporated into the medicament such that it is released sequentially after the release of the catabolic agent.

Alternatively, the anabolic agent can be provided as a separate medicament. In such an embodiment of the invention, the medicament comprising the catabolic agent and the medicament comprising the anabolic agent can be administered to the subject either simultaneously or sequentially. Preferably if administered sequentially the catabolic agent is administered prior to the anabolic agent.

A cartilage pathology is herein defined as any deviation from a healthy, normal, or efficient condition of the cartilage. This encompasses osteoarthritis and focal cartilage defects. A focal cartilage defect can be a symptom of osteoarthritis.

The subject with the cartilage pathology can be a human or a non-human mammal.

Clinically, in open joint surgery to treat focal lesions, the window of opportunity to manipulate cartilage is approximately 2 hours. Advantageously therefore the medicament suitable for administeration to the subject intra-operatively.

The medicament can be provided in a variety of formulations known to the skilled man.

It is envisaged that the medicament can be administered at/or near the site of the cartilage pathology. As such, in specific embodiments of the invention the medicament is formulated in a manner that will enable local administration.

In further specific embodiments of the invention the medicament is formulated in a manner that will enable topical administration to the surface of the cartilage defect. For example, as an irrigation fluid used during surgical procedures, such as arthroscopy.

It is also envisaged that at medicament can be used in a clinical setting, and optionally pre- or post-surgery. For example, the medicament can be administered to a subject presenting with a cartilage pathology by intra-articular injection. Advantageously this may inhibit, delay or reverse the progression of the cartilage pathology thereby preventing the need for surgical intervention.

The administration pattern of the medicament can comprise administering the medicament as a single or multiple dose.

The medicament for intra-articular injection can further comprise cells, for example, mesenchymal stem cells, chondroprogenitor cells or chondrocytes.

It is envisaged in the treatment of a focal defect that the treatment protocol can comprise a single or multiple administration of the medicament to the defect site.

In a particular embodiment of the invention the medicament comprising the at a least one agent capable of the catabolism of at least one component of the extracellular matrix is used during an arthroscopy procedure. For example the surgeon will excise any damaged cartilage from the defect and then administer the medicament at least to the resected area. For example, the medicament may be topically applied to the cartilage using suitable means, for example a swab. Alternatively, the medicament may be provided as a component of the irrigation fluid which is used to clean the defect.

In a particular embodiment of the invention the medicament comprising the at a least one agent capable of the catabolism of at least one component of the extracellular matrix is used during a surgical procedure in which a cartilage implant/cartilage tissue substitute material is implanted into a cartilage defect. In this procedure it is envisaged that the surgeon will excise any damaged cartilage from the defect. Prior to implanting the device the surgeon will administer the medicament to the defect. For example, the medicament may be topically applied to the cartilage using suitable means, for example a swab. Alternatively, the medicament may be provided as a component of the irrigation fluid which is used to clean the defect. Advantageously the medicament is administered to the margins of the defect which will be adjacent to/abut the device in situ. The implant will then either immediately or within a short time period be implanted.

There is provided a surgical irrigant fluid comprising an agent capable of causing the catabolism of a component of cartilage extracellular matrix.

In further embodiments of the invention the surgical irrigant fluid further comprises an agent capable of causing the anabolism of a component of cartilage extracellular matrix.

In a further embodiment of the invention the medicament is alternatively or additionally provided on the cartilage implant/cartilage tissue substitute material.

There is provided an implant comprising an agent capable of causing the catabolism of a component of cartilage extracellular matrix.

In further embodiments of the invention the implant further comprises an agent capable of causing the anabolism of a component of cartilage extracellular matrix.

In embodiments of the invention the implant is a bioresorbable scaffold.

According to a still further aspect of the invention there is provided the uses, methods, medicaments or products as herein described with reference to the accompanying Examples and Figures, in which;

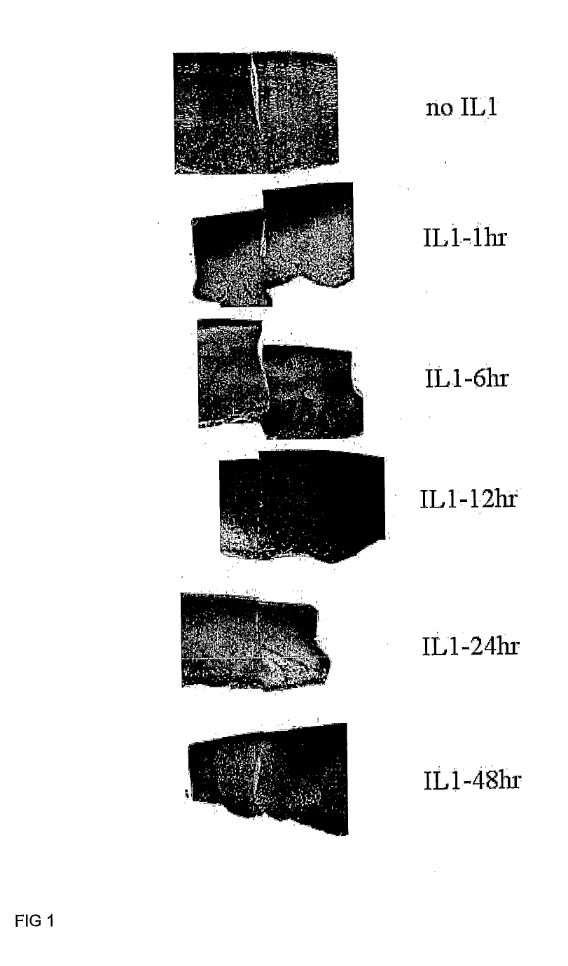

FIG. 1: Illustrates the incubation of explants with IL-1β for varying time periods.

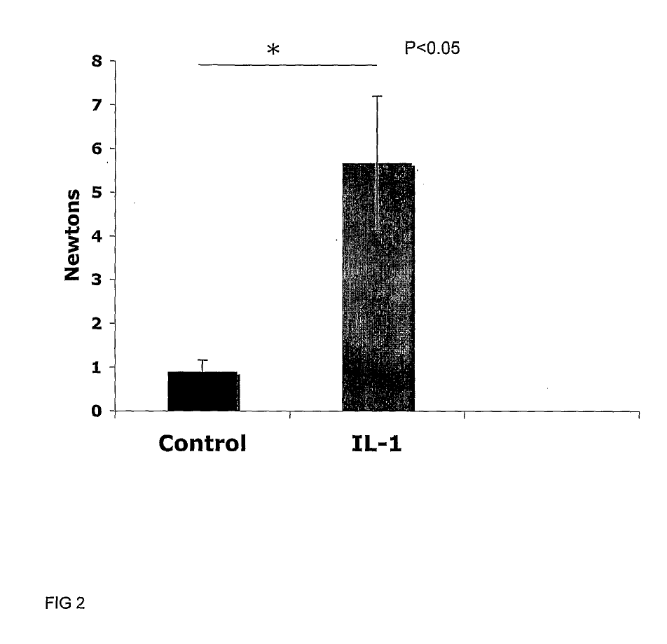

FIG. 2: Mechanical “push-out” tests of IL-1β treated explants

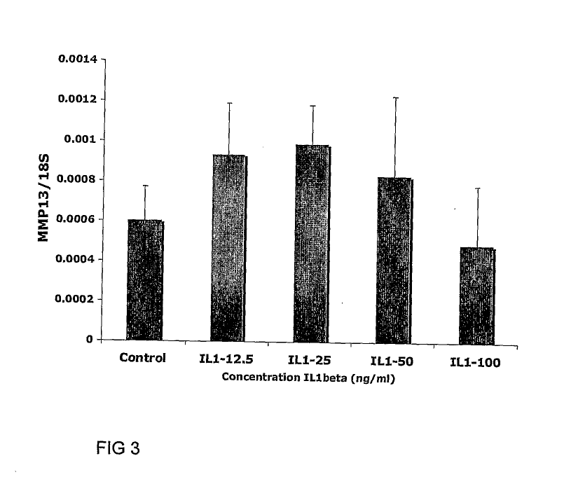

FIG. 3: Gene expression analysis of MMP13 following 2 hour exposure to IL1β.

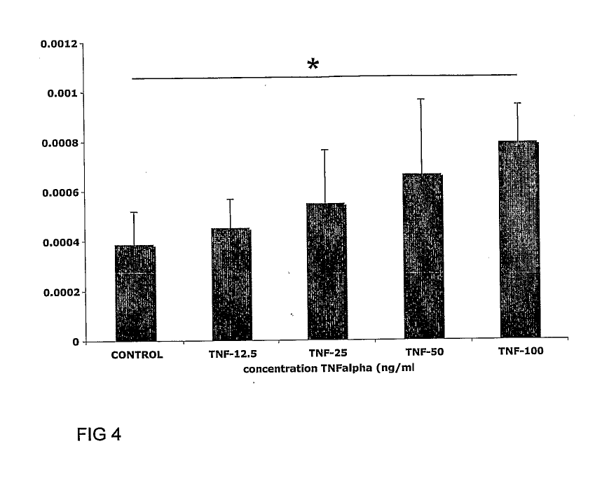

FIG. 4: Gene expression analysis of MMP13 induction following 2 hour exposure to TNFα.

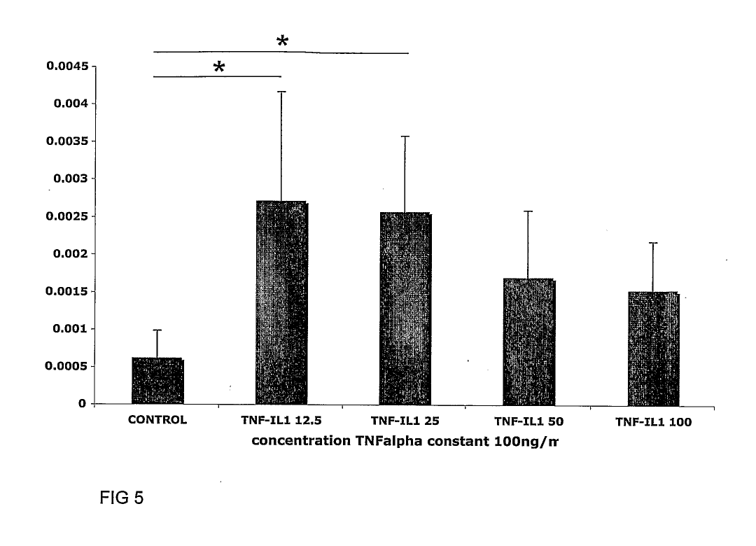

FIG. 5: Gene expression analysis of MMP13 expression following the addition of a constant concentration of TNFα and increasing concentrations of IL1β.

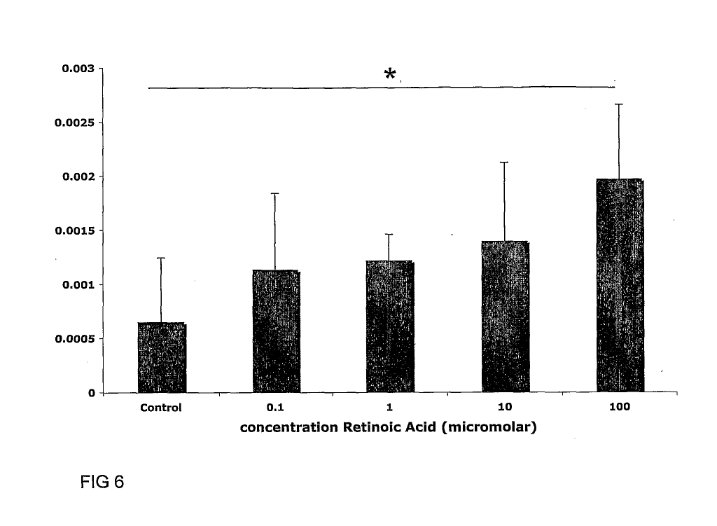

FIG. 6: Gene expression analysis of MMP13 gene induction following exposure to increasing concentrations of retinoic acid.

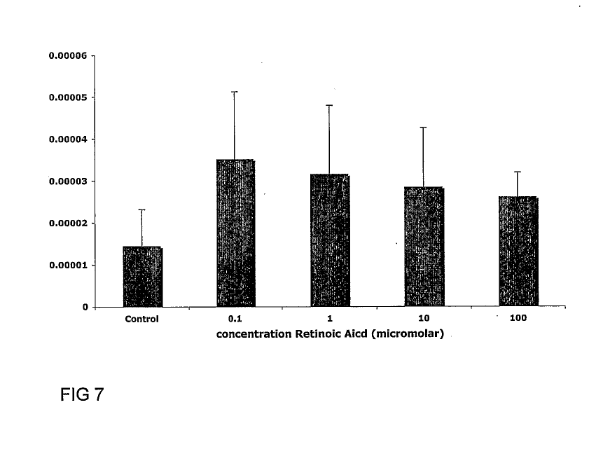

FIG. 7: Gene expression analysis of ADAMTS4 gene induction following exposure to increasing concentrations of retinoic acid.

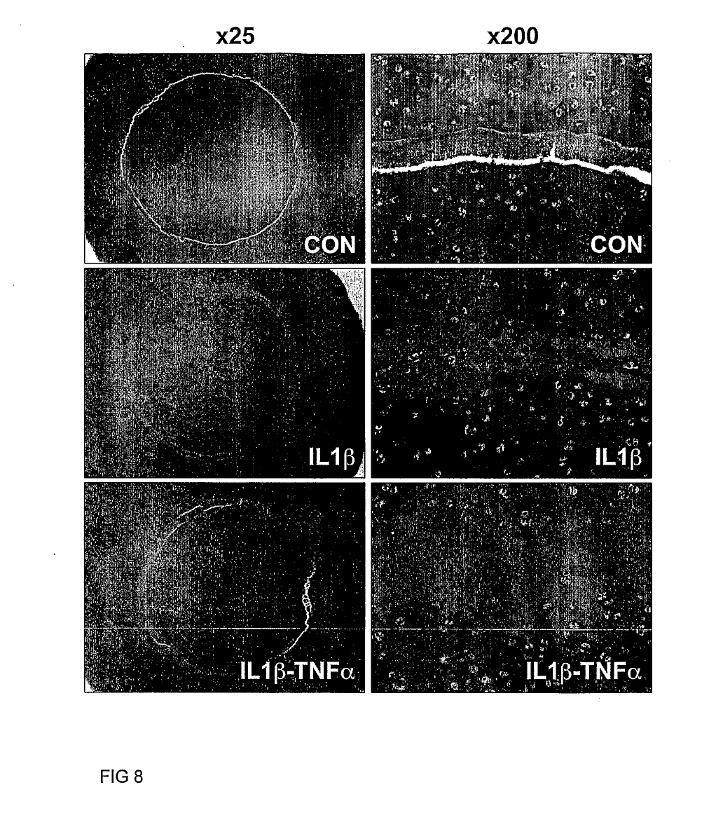

FIG. 8: In vitro culture of control, IL1β and IL1β/INFα treated explants.

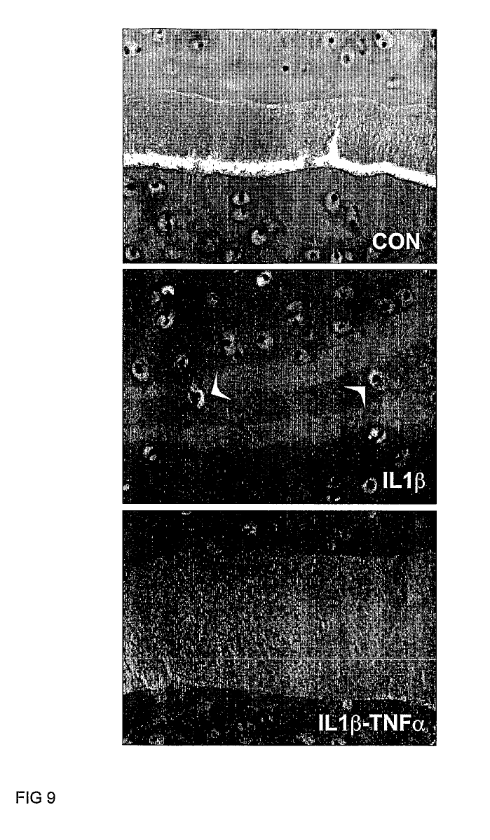

FIG. 9: High power images of FIG. 8 showing the interfacial matrix of cultured explants that were initially treated with cytokines

### SPECIFIC EMBODIMENTS OF THE INVENTION

Materials and Methods

Determination of the Optimum IL-1β Treatment Time for Integration

Six millimetre diameter cartilage explants from immature articular cartilage containing 3 mm diameter inner cores, both created using punch biopsy tools (Steifel), were placed in Dulbecco's modified Eagles medium (DMEM) culture medium containing various concentrations of interleukin-1β or tumour necrosis factor α (TNFα; Peprotech), or, the culture medium without cytokine as a control. The medium was removed after the nominated time and washed once with DMEM then replaced with DMEM medium containing 50 μg/ml ascorbate, 10 mM HEPES, gentamycin and supplemented with 1× insulin-transferrin-selenium (ITS; Sigma). For qPCR studies the explants were first allowed to equilibriate in serum containing medium for 3 days, then washed 3 times in serum-free DMEM before the treatment as described above. For long term culture following 2 hour cytokine treatment explants were treated immediately following their excision from the joint, then cultured for 4 weeks in serum-free DMEM containing ITS, ascorbate, HEPES and gentamycin. The culture medium was changes 3 times a week.

Real-Time PCR Amplification and Quantitative Analysis.

Quantitative polymerase chain reaction (qPCR) using the fluorescent dye SYBR Green (Eurogentec, Belgium) was used to determine the absolute expression levels of ADAMTS4 and MMP-13 between explants. Real-time PCR reactions were carried out in 25 μl volumes in a 96-well plate (Applied Biosystems™) containing 1× buffer (10×), 3.5 mM MgCl₂, 200 μM dNTPs, 0.3 μM of sense and antisense primers, 0.025 U enzyme and 1:66000 SYBR GreenI®. All reactions were made using gPCR™ Core Kit for SYBR Green I® (Eurogentec). At the end of each reaction, the cycle threshold (C_{T}) was manually setup at the level that reflected the best kinetic PCR parameters, and melting curves were acquired and analysed. Absolute values for the gene of interest were calculated from standard curves generated using serially diluted plasmid cloned and sequence verified template (ng DNA) and were normalized to the housekeeping gene 18S rRNA.

Histological Preparation and Staining of Explant Cultures

Four week core and disc cultures were fixed with neutral-buffered formyl-saline and left overnight at 4 C. The explants were then processed for wax embedding. Ten micron sections were cut using a microtome, and sections dewaxed and rehydrated in a descending series of alcohols. Sections were stained with haemotoxylin (1 minute), washed in water and then stained with Safranin-O (2 minutes) washed briefly in water and then dehydrated, dipped in xylene and then mounted in DPX liquid under coverslips.

Mechanical Testing

Adhesive properties of the disc/ring interface after 3-weeks in culture were assessed using a push-out test (n=4). Thickness of the sample was measured using calipers. A custom-made mechanical testing device in which a ‘push-out rod’ displaced the disc from the ring was used to test the adhesive strength with a Lloyd LRX material testing machine (Lloyds Instruments Ltd, Hants, UK). A computer activated micro-stepper controlled the displacement of the push-out rod (0.05 mm/min), whilst a load cell (100N) coupled to the rod measured the push-out force. The adhesive strength was calculated from the maximum force measured at failure per unit of interfacial area.

Results

The Effect of IL-1β on Cartilage Integration

We have shown that in unsupplemented culture medium treatment with 10 ng/ml IL1β for 24 hours enhances integration between disc and core cartilages, that have been cultured for a further 2 weeks FIG. 1A.

“Push-Out” Test

As shown in FIG. 2, explants treated with IL1β required almost six times more force to “push out” the explant from the disc as did untreated explants. This illustrates integration at the interfacial area.

The Effect of IL1β Exposure on MMP13 Gene Expression

Treatment of explants with increasing concentrations of IL1β (12.5-100 ng/ml) for 2 hours resulted in an increase in MMP13 gene expression (FIG. 3). The increase in gene expression was 1.64-fold in samples incubated with 25 ng/ml IL1β (P>0.116; n=3).

The Effect of TNFα Exposure on MMP13 Gene Expression

Treatment of explants with increasing concentrations of TNFα (12.5-100 ng/ml) for 2 hours resulted in a linear increase in MMP13 gene expression. The highest concentration of TNFα elicited a statistically significant increase in gene expression of approximately 2-fold (P<0.03; n=3), (FIG. 4).

The Effect of TNFα and IL1β on MMP13 Gene Expression

Explants were incubated in a constant concentration of TNFα of 100 ng/ml in combination with increasing concentrations of IL1β (12.5-100 ng/ml). The combination of cytokines had a significant effect on MMP13 gene expression increasing it approximately 4-fold (P<0.02; n=3) when IL1β was used at 12.5 ng/ml (FIG. 5).

The Effect of Retinoic Acid on MMP13 and ADAMTS4 Gene Expression

Retinoic acid is a molecule known to have catabolic effects on articular cartilage, principally the induction of metalloproteinase activity. Explants were incubated with increasing concentrations of retinoic acid (0.1-100 μM). A concentration of 100 μM induced an increase of approximately 3-fold in MMP13 gene expression following 2 hour incubation (P<0.03; n=3) (FIG. 6). Through analysis of ADAMTS4 gene expression, it was demonstrated that MMP gene expression was induced to a greater extent than genes that specifically targeted proteoglycan proteolytic activity (FIG. 7).

Disc-Core Explant Cultures Display Differences in Chondrocyte Migration Following IL1β Treatment

Disc-core explants were treated with either 25 ng/ml IL1β or 100 ng/ml TNFα/12.5 ng/ml IL1β for 2 hours and then cultured for a further 4 weeks in serum-free culture medium containing the ITS supplement. Histologic analysis of safranin-O stained explants sectioned tangentially shows that integration, assessed qualitatively through retention of matrix integrity, was best achieved in explants that had been treated with IL1β (FIG. 8), although improved integration was also noted for explants treated with the cytokine combination. At high power it can be observed that part of the mechanism that ensured good integration was the presence of migrating chondrocytes within the interfacial matrix that had been deposited by the opposing surfaces of the disc and core cartilages in IL1β treated cartilages (FIG. 9).

### REFERENCES

Bos, P. K., DeGroot, J., Budde, M., Verhaar, J. A. and van Osch, G. J. (2002). Specific enzymatic treatment of bovine and human articular cartilage: implications for integrative cartilage repair. Arthritis and rheumatism 46, 976-85.
Caplan, A. I., Elyaderani, M., Mochizuki, Y., Wakitani, S. and Goldberg, V. M. (1997). Principles of cartilage repair and regeneration. Clinical orthopaedics and related research, 254-69.
Hunziker, E. B. (2002). Articular cartilage repair: basic science and clinical progress. A review of the current status and prospects. Osteoarthritis and cartilage/OARS, Osteoarthritis Research Society 10, 432-63.
Janssen, L. M., In der Maur, C. D., Bos, P. K., Hardillo, J. A. and van Osch, G. J. (2006). Short-duration enzymatic treatment promotes integration of a cartilage graft in a defect. The Annals of otology, rhinology, and laryngology 115, 461-8.
Obradovic, B., Martin, I., Padera, R. F., Treppo, S., Freed, L. E. and Vunjak-Novakovic, G. (2001). Integration of engineered cartilage. Journal of orthopaedic research 19, 1089-97.
Shapiro, F., Koide, S. and Glimcher, M. J. (1993). Cell origin and differentiation in the repair of full-thickness defects of articular cartilage. The Journal of bone and joint surgery 75, 532-53.
Tetlow, L. C., Adlam, D. J. and Woolley, D. E. (2001). Matrix metalloproteinase and proinflammatory cytokine production by chondrocytes of human osteoarthritic cartilage: associations with degenerative changes. Arthritis and rheumatism 44, 585-94.
van de Breevaart Bravenboer, J., In der Maur, C. D., Bos, P. K., Feenstra, L., Verhaar, J. A., Weinans, H. and van Osch, G. J. (2004). Improved cartilage integration and interfacial strength after enzymatic treatment in a cartilage transplantation model. Arthritis research & therapy 6, R469-76.

1. (canceled)

2. A composition comprising at least one agent capable of the catabolism of at least one component of cartilage extracellular matrix and at least one agent capable of the anabolism of at least one component of cartilage extracellular matrix.

3. A method of treatment of a cartilage pathology in a subject comprising administering a composition comprising at least one agent capable of the catabolism of at least one component of cartilage extracellular matrix to the subject.

4. The method of claim 3 wherein the agent capable of the catabolism of at least one component of cartilage extracellular matrix catabolises the collagen component and/or the proteoglycan component.

5. The method of claim 4 wherein the collagen component is type II collagen.

6. The method of claim 4 wherein the proteoglycan component is chondroitin sulphate.

7. The method of claim 3 wherein the agent capable of the catabolism of at least one component of cartilage extracellular matrix is a cytokine.

8. The method of claim 7 wherein the cytokine is a catabolic cytokine.

9. The method of claim 8 wherein the catabolic cytokine is selected from the group consisting of interleukin-1 alpha (IL-1α), interleukin-1 beta (IL-1β), tumor necrosis factor alpha (TNFα), oncostatin M and isoforms thereof.

10. The method of claim 3 wherein the agent capable of the catabolism of at least one component of cartilage extracellular matrix comprises at least two catabolic cytokines.

11. The method of claim 3 wherein the agent capable of the catabolism of at least one component of cartilage extracellular matrix comprises interleukin-1 beta (IL-1β) and tumor necrosis factor alpha (TNFα).

12. The method of claim 11 wherein the interleukin-1 beta (IL-1β) is at a therapeutically effective dose equal to or greater than about 100 pg/ml.

13. The method of claim 11 wherein the interleukin-1 beta (IL-1β) is at a therapeutically effective dose equal to or less than about 10 ng/ml.

14. The method of claim 3 wherein the agent capable of the catabolism of at least one component of cartilage extracellular matrix is a proteolytic enzyme.

15. The method of claim 14 wherein the proteolytic enzyme is a matrix metalloproteinase (MMP).

16. The method of claim 15 wherein the proteolytic enzyme is a disintegrin and metalloproteinase (ADAM).

17. The method of claim 3 wherein the agent capable of the catabolism of at least one component of cartilage extracellular matrix is an activator of the pro-form of a proteolytic enzyme.

18. The method of claim 3 wherein the composition further comprises an agent capable of the anabolism of at least one component of the extracellular matrix.

19. The method of claim 18 wherein the agent capable of the anabolism of at least one component of the extracellular matrix is an anabolic, modulatory or non-catabolic cytokine.

20. The method of claim 19 wherein the agent is a bone morphogenetic protein (BMP).

21. An implant comprising an agent capable of causing the catabolism of a component of cartilage extracellular matrix.

22. The implant of claim 21 wherein the implant further comprises an agent capable of causing the anabolism of a component of cartilage extracellular matrix.

23. A surgical irrigant fluid comprising an agent capable of causing the catabolism of a component of cartilage extracellular matrix.

24. The surgical irrigant fluid of claim 23 wherein the surgical irrigant fluid further comprises an agent capable of causing the anabolism of a component of cartilage extracellular matrix.
